(19) United States
(12) Patent Application Publication
(10) Pub. No.: US 2016/0024504 A1
(43) Pub. Date: Jan. 28, 2016

(54) TREATING TH2-MEDIATED DISEASES BY INHIBITION OF BROMODOMAINS

(71) Applicants: GENENTECH, INC., South Francisco, CA (US); CONSTELLATION PHARMACEUTICALS, INC., Cambridge, MA (US)

(72) Inventors: Brian K. Albrecht, Cambridge, MA (US); Alexandre Cote, Cambridge, MA (US); Terry Crawford, South San Francisco, CA (US); Benjamin Fauber, South San Francisco, CA (US); Hon-Ren Huang, Cambridge, MA (US); Jose M. Lora, Cambridge, MA (US); Steven Magnuson, South San Francisco, CA (US); Christopher G. Nasveschuk, Cambridge, MA (US); Andres Salmeron, Cambridge, MA (US); Robert J. Sims, III, Cambridge, MA (US); Alexander M. Taylor, Cambridge, MA (US)

(73) Assignees: CONSTELLATION PHARMACEUTICALS, INC., Cambridge, MA (US); GENENTECH, INC., South San Francisco, CA (US)

(21) Appl. No.: 14/776,051

(22) PCT Filed: Mar. 14, 2014
(86) PCT No.: PCT/US14/29252

§ 371 Date: Sep. 14, 2015

Related U.S. Application Data
(60) Provisional application No. 61/798,644, filed on Mar. 15, 2013.

(51) Int. Cl. C12N 15/113; G01N 33/50; A61K 31/444; A61K 31/437; A61K 31/496

(52) U.S. Cl. CPC C12N 15/113; A61K 31/437; A61K 31/496; A61K 31/444; G01N 33/505; C12N 2310/14; C12N 2320/30; G01N 2500/10; G01N 2500/04; G01N 2333/52

(57) ABSTRACT

The invention provides methods for treating Th2 cytokine-mediated diseases by inhibiting bromodomain function.

### CROSS-REFERENCE TO RELATED APPLICATION(S)

This patent application claims the benefit of priority of U.S. application Ser. No. 61/798,644, filed Mar. 15, 2013, which application is herein incorporated by reference.

### BACKGROUND

Chromatin is a complex combination of DNA and protein that makes up chromosomes. It is found inside the nuclei of eukaryotic cells and is divided between heterochromatin (condensed) and euchromatin (extended) forms. The major components of chromatin are DNA and proteins. Histones are the chief protein components of chromatin, acting as spools around which DNA winds. The functions of chromatin are to package DNA into a smaller volume to fit in the cell, to strengthen the DNA to allow mitosis and meiosis, and to serve as a mechanism to control expression and DNA replication. The chromatin structure is controlled by a series of post translational modifications to histone proteins, notably histones H3 and H4, and most commonly within the “histone tails” which extend beyond the core nucleosome structure. Histone tails tend to be free for protein-protein interaction and are also the portion of the histone most prone to post-translational modification. These modifications include acetylation, methylation, phosphorylation, ubiquitinylation, SUMOylation. These epigenetic marks are written and erased by specific enzymes, which place the tags on specific residues within the histone tail, thereby forming an epigenetic code, which is then interpreted by the cell to allow gene specific regulation of chromatin structure and thereby transcription.

Of all classes of proteins, histones are amongst the most susceptible to post-translational modification. Histone modifications are dynamic, as they can be added or removed in response to specific stimuli, and these modifications direct both structural changes to chromatin and alterations in gene transcription. Distinct classes of enzymes, namely histone acetyltransferases (HATS) and histone deacetylases (HDACs), acetylate or de-acetylate specific histone lysine residues (Struhl K., Genes Dev., 1989, 12, 5, 599-606).

Bromodomains, which are approximately 110 amino acids long, are found in a large number of chromatin-associated proteins and have been identified in approximately 70 human proteins, often adjacent to other protein motifs (Jeanmougin F., et al., Trends Biochem. Sci., 1997, 22, 5, 151-153; and Tamkun J. W., et al., Cell, 1992, 7, 3, 561-572). Interactions between bromodomains and modified histones may be an important mechanism underlying chromatin structural changes and gene regulation. Bromodomain-containing proteins have been implicated in disease processes including cancer, inflammation and viral replication.

There is currently a need for methods for treating diseases associated with the TH2 cytokine, such as immune related diseases, allergic diseases, respiratory disorders, and eosinophil associated diseases.

### SUMMARY

As described herein, it has been demonstrated that BRD7 and/or BRD9 bromodomains play an unexpected and important role in the expression of Th2 cytokines, in particular IL-4, IL-5 and IL-13. Accordingly, the present invention provides a method for treating a TH2 disease in a mammal comprising administering a therapeutically effective amount of an inhibitor of BRD7 and/or BRD9 to the mammal.

The invention also provides an inhibitor of BRD7 and/or BRD9 for the prophylactic or therapeutic treatment of a TH2 disease.

The invention also provides the use of an inhibitor of BRD7 and/or BRD9 to prepare a medicament for the treatment of a TH2 disease.

The invention also provides a pharmaceutical composition for use in the treatment of a TH2 disease, comprising an inhibitor of BRD7 and/or BRD9 and a pharmaceutically acceptable carrier.

The invention also provides a method of identifying a compound useful for treating a TH2 disease comprising determining whether the compound inhibits BRD7 and/or BRD9.

The invention also provides a method for inhibiting the production of IL4, IL5, or IL13 in a mammal comprising administering an inhibitor of BRD7 and/or BRD9 to the mammal.

The invention also provides for a method of treating a TH2 disease mediated by IL-4, IL-5, and/or IL-13 (that is, an IL-4 mediated disease, an IL-5 mediated disease, or an IL-13 mediated disease).

### BRIEF DESCRIPTION OF THE FIGURES

FIG. 1. Human T Blasts were stimulated for 48 hours using anti-CD3 and anti-CD28 antibodies in the presence of DMSO or different concentrations of three BRD7/9 inhibitory compounds. A control of unstimulated cells in the presence of DMSO was also included. Cell supernatants were collected at the end of stimulation and cytokine levels were measured using the Luminex platform. Cell viability was determined using the Cell Titer-Glo kit (Promega). FIG. 1a shows Cell Titer Glo (CTG) data plus data on IL-4, IL-5, TNF, IFN-γ and IL-17F levels. FIG. 1b shows data on nine additional cytokines.

FIG. 2. Human naïve T cells (CD4+CD45RA+) cells isolated from peripheral blood of healthy volunteers were polarized into Th2 cells in vitro. After six days of differentiation, cells were washed and re-stimulated for 40 hours using anti-CD3 and anti-CD28 antibodies in the presence of DMSO or different concentrations of compound BRD7/9 (3) and BRD7/9 (4). A control of unstimulated cells in the presence of DMSO was also included. Cell Titer Glo (CTG) and cytokine levels were measured in supernatants using the Luminex platform. FIG. 2a shows CTG data together with IL-5 and TNF data. FIG. 2b shows data on nine additional cytokines.

FIG. 3. Human naïve T cells (CD4+CD45RA+) cells isolated from peripheral blood of healthy volunteers were polarized into Th2 cells in vitro. After six days of differentiation, cells were washed and re-stimulated for 40 hours using anti-CD3 and anti-CD28 antibodies in the presence of DMSO or different concentrations of compound BRD7/9 (3) and BRD7/9 (4). A control of unstimulated cells in the presence of DMSO was also included. IL-5 levels were measured in supernatants using the Luminex platform and AlphaLISA detection method.

FIG. 4. Human naïve T cells (CD4+CD45RA+) cells isolated from peripheral blood of healthy volunteers were polarized into Th2 cells in vitro. After six days of differentiation, cells were washed and re-stimulated for 24 hours using anti-CD3 and anti-CD28 antibodies in the presence of DMSO or 1 uM concentration of compounds BRD7/9 (4), BRD7/9 (5) or BRD7/9 (6). A control of unstimulated cells in the presence of DMSO was also included. Levels of IL-5 and IL-13 mRNA were calculated using RT-PCR standardized to the levels of GAPDH in each sample. Fold induction calculated against control unstimulated cells (DMSO (−)).

FIG. 5. Human naïve T cells (CD4+CD45RA+) cells isolated from peripheral blood of healthy volunteers were polarized into Th2 cells in vitro. After six days of differentiation, cells were washed and re-stimulated for 40 hours using anti-CD3 and anti-CD28 antibodies in the presence of DMSO or different concentrations of eight BRD7/9 compounds with diverse biochemical potencies. A control of unstimulated cells in the presence of DMSO was also included. IL-5 and IL-13 levels were measured in supernatants using AlphaLISA detection method.

FIG. 6. Human naïve T cells (CD4+CD45RA+) cells isolated from peripheral blood of healthy volunteers were polarized into Th1 and Th17 cells in vitro. After six days of differentiation, cells were washed and re-stimulated for 40 hours using anti-CD3 and anti-CD28 antibodies in the presence of DMSO or different concentrations of compound BRD7/9 (3) and BRD7/9 (4). A control of unstimulated cells in the presence of DMSO was also included. IFN-γ and IL-17A levels were measured in supernatants using the Luminex platform. CTG determined as a measure of cell viability.

FIG. 7. Human naïve T cells (CD4+CD45RA+) cells isolated from peripheral blood of healthy volunteers were polarized into Th1, Th2, Th17 or Treg cells in vitro in the presence of DMSO or 1 uM concentrations of compounds BRD7/9 (3) or BRD7/9 (4). After seven days of differentiation, cells were washed and re-stimulated for 6 hours using PMA/Ionomycin+GolgiPlug. Intracellular cell staining with specific labeled antibodies followed by FACS was performed. Data analyzed and plotted using Flojo software.

FIG. 8. Mouse naïve T cells (CD4+CD62L+) cells isolated from spleens of BalbC mice were polarized into Th2 cells in vitro. After four days of differentiation, cells were washed and re-stimulated for 40 hours using anti-CD3 and anti-CD28 antibodies (dynabeads) in the presence of DMSO or different concentrations of compound BRD7/9 (3), BRD7/9 (4), BRD7/9 (6) and BRD7/9 (8). A control of unstimulated cells in the presence of DMSO was also included. Cell Titer Glo (CTG) and cytokine levels were measured in supernatants using the Luminex platform.

FIG. 9. The sequence of isoform 1 was used to generate the recombinant bromodomain protein for both BRD7 (SEQ ID NO:1) and BRD9 (SEQ ID NO:2). For BRD7, the portion of protein used in the DSF assay begins at line 3, residues EEV and ends at line 4, residues QER. For BRD9, the portion used begins at line 3, residues AEN and ends at line 4, residues MSK.

FIGS. 10A-10C. Relocalization of BRD9 upon inhibitor treatment. Visible areas are individual nuclei shown at 180× magnification. A. DMSO control. B. Treatment with 10 μM compound “A”. C. Treatment with 10 μM compound BRD7/9 (1).

FIG. 11. Dose-response curves for relocalization of BRD9 upon treatment with BRD7/9 (8).

### DETAILED DESCRIPTION

### Definitions

As used herein “BRD7” includes at least isoform 1 of BRD7 and/or any of its isoforms or naturally occurring variants that comprise a bromodomain. Bromodomains are known as protein domains that bind acetylated lysine residue(s). Human BRD7 isoform 1 comprises the following amino acid sequence of Q9NPI1-1 (UniprotKB/Swiss Prot uniprot.org/uniprot/Q9NPI1.

As used herein “BRD9” includes at least isoform 1 of BRD9 and/or any of its isoforms or naturally occurring variants that comprise a bromodomain. As noted above, bromodomains are known as protein domains that bind acetylated lysine residue(s). Human BRD9 isoform 1 comprises the following amino acid sequence of Q9H8M2-5 (UniprotKB/Swiss Prot-uniprot.org/uniprot/Q9H8M2.

In one preferred embodiment of inhibition, the inhibitor binds to isoform 1 of BRD7 and/or BRD9. In yet another preferred embodiment, the inhibitor binds to isoform 1 of human BRD7 and/or 9.

A “TH2 disease” as used herein is an immune-related disease or disorder associated with excess TH2 cytokine and/or TH2 cytokine activity in which atypical symptoms may manifest due to the levels or activity of the TH2 cytokine locally and/or systemically in the body. Such TH2 cytokines may by expressed by TH2 cells or other cell types such as innate lymphoid cells. A TH2 cytokine as used herein is any one or combination of the following: IL-4, IL-5 and IL-13. In certain embodiments, a TH2 disease is a respiratory disorder or an eosinophilic disorder. Examples of TH2 diseases include: atopic dermatitis, allergies, allergic rhinitis, asthma, fibrosis (including idiopathic pulmonary fibrosis), chronic obstructive pulmonary disease (COPD), hypereosinophilic syndrome, eosinophilic esophagitis, Churg-Strauss syndrome, and nasal polyposis.

An “IL-4 mediated disease” means: a disease associated with excess IL-4 levels or activity in which atypical symptoms may manifest due to the levels or activity of IL-4 locally and/or systemically in the body. Examples of IL-4 mediated diseases include: cancers (e.g., non-Hodgkin's lymphoma, glioblastoma), atopic dermatitis, allergic rhinitis, asthma, fibrosis, lung inflammatory disorders (e.g., pulmonary fibrosis such as IPF), COPD, and hepatic fibrosis.

An “IL-5 mediated disease” means: a disease associated with excess IL-5 levels or activity in which atypical symptoms may manifest due to the levels or activity of IL-5 locally and/or systemically in the body. Examples of IL-5 mediated diseases include: cancers (e.g., non-Hodgkin's lymphoma, glioblastoma), atopic dermatitis, allergic rhinitis, asthma, fibrosis, lung inflammatory disorders (e.g., pulmonary fibrosis such as IPF), COPD, and hepatic fibrosis.

An “IL-13 mediated disease” means a disease associated with excess IL-13 levels or activity in which atypical symptoms may manifest due to the levels or activity of IL-13 locally and/or systemically in the body. Examples of IL-13 mediated diseases include: cancers (e.g., non-Hodgkin's lymphoma, glioblastoma), atopic dermatitis, allergic rhinitis, asthma, fibrosis, lung inflammatory disorders (e.g., pulmonary fibrosis such as IPF), COPD, and hepatic fibrosis.

The term “respiratory disorder” include, but is not limited to asthma; bronchitis (e.g., chronic bronchitis); chronic obstructive pulmonary disease (COPD) (e.g., emphysema (e.g., cigarette-induced emphysema)); conditions involving airway inflammation, eosinophilia, fibrosis and excess mucus production, e.g., cystic fibrosis, pulmonary fibrosis (e.g., idiopathic pulmonary fibrosis), and allergic rhinitis. Examples of diseases that can be characterized by airway inflammation, excessive airway secretion, and airway obstruction include asthma, chronic bronchitis, bronchiectasis, and cystic fibrosis.

The term “eosinophilic disorder” means: a disorder associated with excess eosinophil numbers in which atypical symptoms may manifest due to the levels or activity of eosinophils locally or systemically in the body. Disorders associated with excess eosinophil numbers or activity include but are not limited to, asthma (including aspirin sensitive asthma), atopic asthma, atopic dermatitis, allergic rhinitis (including seasonal allergic rhinitis), non-allergic rhinitis, asthma, severe asthma, chronic eosinophilic pneumonia, allergic bronchopulmonary aspergillosis, coeliac disease, Churg-Strauss syndrome (periarteritis nodosa plus atopy), eosinophilic myalgia syndrome, hypereosinophilic syndrome, oedematous reactions including episodic angioedema, helminth infections, where eosinophils may have a protective role, onchocercal dermatitis and Eosinophil-Associated Gastrointestinal Disorders, including but not limited to, eosinophilic esophagitis, eosinophilic gastritis, eosinophilic gastroenteritis, eosinophilic enteritis and eosinophilic colitis, nasal micropolyposis and polyposis, aspirin intolerance, asthma and obstructive sleep apnoea. Eosinophil-derived secretory products have also been associated with the promotion of angiogenesis and connective tissue formation in tumours and the fibrotic responses seen in conditions such as chronic asthma, scleroderma and endomyocardial fibrosis (Munitz A, Levi-Schaffer F. Allergy 2004; 59: 268-75, Adamko et al. Allergy 2005; 60: 13-22, Oldhoff, et al. Allergy 2005; 60: 693-6). Other examples include cancer (e.g., glioblastoma (such as glioblastoma multiforme), non-Hodgkin's lymphoma (NHL)), atopic dermatitis, allergic rhinitis, asthma, fibrosis, pulmonary fibrosis (including idiopathic pulmonary fibrosis (IPF) and pulmonary fibrosis secondary to sclerosis), COPD, hepatic fibrosis.

“Inhibitor” as used herein includes any compound or treatment capable of inhibiting the expression and/or function of a given bromodomain-containing protein (e.g. a BRD7 or BRD9 containing protein), including any compound or treatment that inhibits transcription of the gene, RNA maturation, RNA translation, post-translational modification of the protein, binding of the protein to an acetylated lysine target (e.g., such as in an inhibition assay as described in Example 1 herein) and the like. Accordingly, “inhibiting the bromodomain-containing protein BRD7” includes inhibiting the expression and/or function of the bromodomain-containing protein BRD7. Similarly, “inhibiting the bromodomain-containing protein BRD9” includes inhibiting the expression and/or function of the bromodomain-containing protein BRD9. For example, in certain embodiments, the inhibitor detectably inhibits the expression level or biological activity of the bromodomain-containing protein as measured, e.g., using an assay described herein. In certain embodiments, the inhibitor inhibits the expression level or biological activity of the bromodomain-containing protein by at least 5%, at least 10%, at least 20%, at least 50%, at least 75%, or at least 90%. The inhibitor may inhibit the production of IL-4, IL-5, and/or IL-13 by inhibiting the expression and/or function of a given bromodomain-containing protein (e.g. a BRD7 or BRD9 containing protein).

The inhibitor can be of natural or synthetic origin. For example, it can be a nucleic acid, a polypeptide, a protein, a peptide, or an organic compound. In one embodiment the inhibitor is an siRNA, shRNA, a small molecule, or a macrocycle.

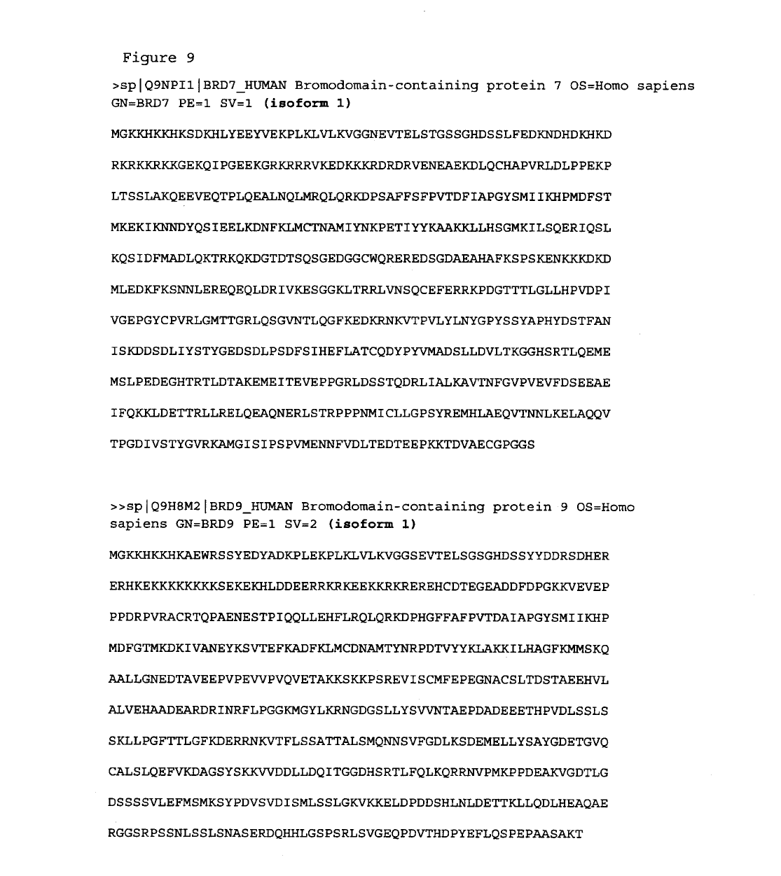

BRD9 inhibitors will in general bind to the acetyllysine binding site of the BRD9 bromodomain and inhibit binding of the protein to acetyllysine or acetyllysine-modified peptides. Residues of BRD9 predicted to be in contact with acetyllysine include (but are not limited to) Va1165, Ala170, Tyr173, Ala212, Asn216, and Tyr222, with residue numbering according to SwissProt entry Q9H8M2 (FIG. 9). Residue Asn216 is of particular importance, and it is expected that BRD9 inhibitors will interact with Asn216. Likewise, BRD7 inhibitors will interact with the acetyllysine binding site of the BRD7 bromodomain. Residues from BRD7 predicted to be in contact with acetyllysine include (but are not limited to) Va1160, Ala165, Tyr168, Ala207, Asn211, and Tyr217, with residue numbering according to SwissProt entry Q9NPI1 (FIG. 9). Residue Asn211 is of particular importance, and it is expected that BRD7 inhibitors will interact with Asn211.

In one embodiment the inhibitor selectively binds to a specific bromodomain-containing protein. For example, the inhibitor may be at least 5, at least 10, at least 50, at least 100, at least 500, or at least 1,000 fold selective for a given bromodomain-containing protein over other bromodomain-containing proteins in a selected assay (e.g., an assay described in the Example 3 herein). In one embodiment the inhibitor may be at least 5, at least 10, at least 50, at least 100, at least 500, or at least 1,000 fold selective for bromodomain-containing protein BRD7 over other bromodomain-containing proteins. In one embodiment the inhibitor may be at least 5, at least 10, at least 50, at least 100, at least 500, or at least 1,000 fold selective for bromodomain-containing protein BRD9 over other bromodomain-containing proteins. In one embodiment the inhibitor may be at least 5, at least 10, at least 50, at least 100, at least 500, or at least 1,000 fold selective for bromodomain-containing proteins BRD7 and BRD9 over other bromodomain-containing proteins. Non-limiting examples of other bromodomain-containing proteins include ASH1L, ATAD2, ATAD2B, BAZ1A, BAZ1B, BAZ2A, BAZ2B, BPTF, BRD1, BRD2, BRD3, BRD4, BRD7, BRD8, BRD9, BRDT, BRPF1, BRPF3, BRWD1, BRWD3, CECR2, CREBBP (aka, CBP), EP300, GCN5L2, KIAA2026, MLL, MLL4, PBRM, PCAF, PHIP, SMARCA2, SMARCA4, SP100, SP110, SP140, SP140L, TAF1, TAF1L, TRIM24, TRIM28, TRIM33, TRIM66, ZMYND8, and ZMYND11. When a protein contains more than one bromodomain, selectivity may be measured against each bromodomain.

In certain embodiments, the inhibitor has an IC₅₀ against BRD7 and/or BRD9 of less than 10 μM, e.g., less than 1 μM, e.g., less than 100 nM, e.g., less than 10 nM, e.g., less than 1 nM.

In certain embodiments, the inhibitor has a binding affinity against BRD7 and/or BRD9 with a K_{d} of less than 1,000 nm, e.g., less than 500 nM, e.g., less than 100 nM, e.g., less than 50 nM. In certain embodiments, the inhibitor has a binding affinity against BRD7 and/or BRD9 of between 500 nM to 1 pM.

In one embodiment the inhibitor is an antisense nucleic acid capable of inhibiting transcription of the bromodomain-containing protein or translation of the corresponding messenger RNA. The anti-sense sequence can be DNA RNA (e.g. siRNA or shRNA), a ribosome, etc. It may be single-stranded or double-stranded. It can also be an RNA encoded by an antisense gene. Using commercially available software, an art worker can design siRNA molecules based on the gene sequences of BRD7 or BRD9.

In one embodiment the inhibitor can be a polypeptide, for example, a peptide containing a region of the bromodomain-containing protein. The polypeptide can also be an antibody against the bromodomain-containing protein, or a fragment or derivative thereof, such as a Fab fragment, a CDR region, or a single chain antibody.

The term “small molecule” includes organic molecules having a molecular weight of less than about 1000 amu. In one embodiment a small molecule can have a molecular weight of less than about 800 amu. In another embodiment a small molecule can have a molecular weight of less than about 500 amu.

Small molecules that may be used in certain embodiments of the invention include the following compounds:

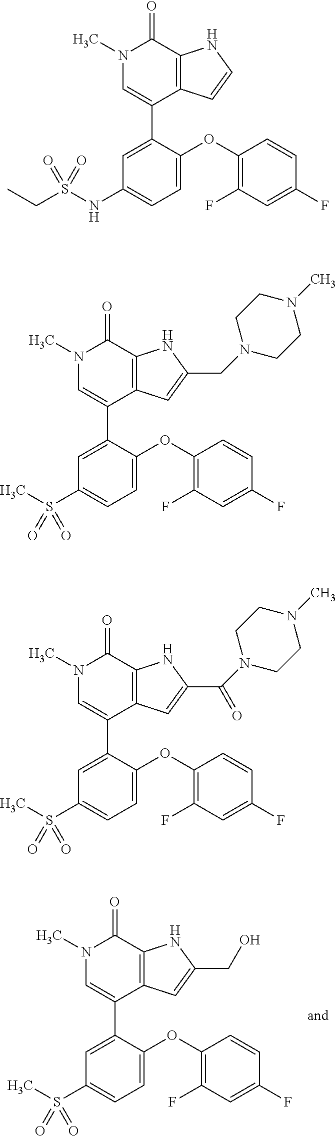

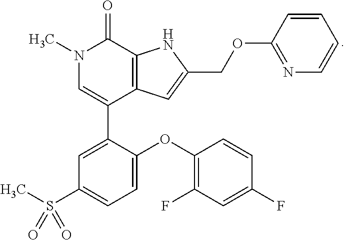 [SMILES: Cn1cc(-c2cc(S(C)(=O)=O)ccc2Oc2ccc(F)cc2F)c2cc(COc3ccccn3)[nH]c2c1=O]

Synthetic intermediates and processes that can be used to prepare these small molecules are described in International Patent Application Publication Number WO 2013/097601.

The term “macrocycle” includes organic molecules having a ring containing nine or more atoms. In one embodiment the macrocycle has a ring containing nine to about 24 atoms. In another embodiment the macrocycle has a ring containing about 12 to about 16 atoms. Typically macrocycles have a molecular weight of less than about 1200 amu. In one embodiment a macrocycle has a molecular weight of less than about 1000 amu. In another embodiment macrocycle has a molecular weight of less than about 800 amu.

As used herein, “treatment” (and grammatical variations thereof such as “treat” or “treating”) refers to clinical intervention to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

In cases where an inhibitor is sufficiently basic or acidic, administration of a pharmaceutically acceptable salt of an inhibitor may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The inhibitors can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the inhibitors may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the inhibitor may be combined with one or more excipients and used in the form of ingestible tablets, buccal tablets, troches, capsules, elixirs, suspensions, syrups, wafers, and the like. Such compositions and preparations should contain at least 0.1% of inhibitor. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The inhibitor may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the inhibitor in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the inhibitors may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver inhibitors to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Certain embodiments of the present invention provide the use of an RNAi molecule as an inhibitor molecule. RNAi molecules include siRNAs, shRNAs, microRNAs (miRNAs) and other small RNA molecules that specifically inhibit protein expression from a target gene, e.g., by causing the destruction of specific mRNA molecules. In certain embodiments, the RNAi molecule targets BRD7 and/or BRD9, e.g., isoform 1 of BRD7 and/or BRD9. In certain embodiments, the RNAi molecule targets human BRD7 and/or BRD9. Using commercially available software, an art worker can design RNAi molecules (e.g., siRNA molecules) based on the gene sequences of BRD7 or BRD9. The RNAi molecule may be delivered (e.g., administered) to a subject in need of treatment using methods known in the art, such as by transfection, electroporation, or viral transfer.

Useful dosages of inhibitors can be determined by comparing their in vitro activity, and in vivo activity in animal models. Methods for the extrapolation of effective dosages in mice, and other animals, to humans are known to the art; for example, see U.S. Pat. No. 4,938,949.

The amount of an inhibitor required for use in treatment will vary not only with the particular salt selected but also with the route of administration, the nature of the condition being treated and the age and condition of the patient and will be ultimately at the discretion of the attendant physician or clinician.

The inhibitor is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising an inhibitor formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The invention will now be illustrated by the following non-limiting Examples.

### Example 1

### Inhibition Studies

### Materials and Methods

T-Blasts Preparation and Re-Stimulation

Peripheral Mononuclear Blood Cells (PBMCs) were isolated from peripheral blood of healthy volunteers using ficoll (GE Biosciences) density gradient centrifugation. Cells were cultured using RPMI (Glutamax) (Invitrogen) containing 10% FBS and Pen/Strep at 1E6 cells/ml with the addition of 10 ug/ml of PHA-P (Sigma L8754) for 4 days. After 4 days, cells were pooled washed and re-seeded at 1E6 cells/ml for 1 day in RPMI media, PHA-P (10 ug/ml) and rhIL-2 (R&D Biosystems (202-IL) (4 ng/ml). Cells were washed and frozen for re-stimulation. Re-stimulation was done at 1E6 cells/ml in DMEM (Glutamax)(Invitrogen) containing 10% FBS and Pen/Strep. 2E5 cells were used per determination point. Cells were stimulated in the presence of DMSO (0.5%) or compounds (0.5% DMSO final) with 5 ug/ml of anti-CD3 (BD Bioscience 555336) and 5 ug/ml of anti-CD28 (BD Bioscience 555726) for 48 hours. Cytokine levels were measured using Luminex platform (Millipore) or AlphaLISA platform (Perkin Elmer) using manufacturer specifications.

Isolation of Human Naïve T Cells (CD4+CD45RA+) and Polarizing Conditions

Peripheral Mononuclear Blood Cells (PBMCs) were isolated from peripheral blood of healthy volunteers using ficoll (GE Biosciences) density gradient centrifugation. CD4+CD45RA+T cells were isolated from PBMCs by magnetic depletion of non-T helper cells and memory CD4+T cells using the human naïve CD4+T Cell Isolation Kit II (130-094-131; Miltenyi Biotech). For the induction of TH17 differentiation, naïve CD4 T cells were activated using Human T-Activator CD3/CD28 Dynabeads® (Invitrogen) and cultured in DMEM (Invitrogen) in the presence of the following cocktail: TGF-beta (10 ng/mL; R&D Biosystems), IL-6 (10 ng/mL; R&D Biosystems), IL-23 (10 ng/mL; R&D Biosystems), IL-1beta (10 ng/mL; R&D Biosystems), anti-human IFN-gamma (10 ug/mL; clone B140; eBioscience) and anti-human IL-4 (10 ug/mL; clone 8D4-8; eBioscience) for 6 days. For TH1 conditions, the following cocktail was used: IL-12 (10 ng/ml; R&D Biosystems), IL-2 (20 ng/ml; R&D Biosystems), anti-human IL-4 (10 ug/mL; clone 8D4-8; eBioscience). TH2 conditions were IL-4 (20 ng/ml; R&D Biosystems), IL-2 (10 ng/ml, R&D Biosystems), anti-human IFN-gamma (10 ug/mL; clone B140; eBioscience) and anti-human p40 (5 ug/ml; clone C8.4 eBiosciences). For iTreg, the following was used: TGF-beta(20 ng/mL; R&D Biosystems), IL-2 (10 ng/ml; R&D Biosystems). Naïve CD4 T cells were polarized under respective conditions for 6-8 days.

Isolation of Mouse Naïve T Cells (CD4+CD62L+) and Polarizing Conditions

CD4+CD62L+naïve T cells were isolated from spleens of 6-8 weeks old female BalbC mice. Single cell suspensions of splenocytes were prepared using 70-μm nylon cell strainers (BD Bioscience). Red blood cells were lysed using ammonium chloride lysis buffer (R7757; Sigma) and washed with cRPMI 10% FBS (61870-036; Invitrogen). Naïve CD4 T cells were purified using magnetic-activated cell sorting beads (130-093-227; Miltenyi Biotec). Purity of sorted naive cells was greater than 90%. Naïve CD4 T cells were cultured in 6-well plates (1×106 cells/ml) and stimulated with anti-CD3/CD28 coated beads (Dynabeads 11452D; Invitrogen) for 4 days under TH2, polarizing conditions: IL-4 10 ng/ml (214-14; Pepro), IL-2 10 ng/ml (402-ML; R&D Biosystems), 10 μg/ml anti-IFN-γ antibody (554408; BD Pharmingen) and 5 ug/ml anti-IL-12 antibody (554475; BD Pharmingen).

Cell Viability

Cell viability was assessed using Cell Titre Glo®, which determines the number of viable cells based on quantitation of ATP present (G7572; Promega).

Re-Stimulation of Human Th2 Cells

Th2 cells differentiated for 6 days were washed and left in incubator ON with normal media DMEM (Glutamax)(Invitrogen) containing 10% FBS and Pen/Strep. The following day, 2E5 cells were used per determination point. Cells stimulated in the presence of DMSO (0.5%) or compounds (0.5% DMSO final) with 5 ug/ml of anti-CD3 (BD Bioscience 555336) and 5 ug/ml of anti-CD28 (BD Bioscience 555726) for 24 or 48 hours. Cytokine levels measured using Luminex platform (Millipore) or AlphaLISA platform (Perkin Elmer) using manufacturer specifications.

Re-Stimulation of Mouse Th2 Cells

Th2 cells differentiated for 4 days were washed and left in incubator ON with normal media RPMI (Glutamax)(Invitrogen) containing 10% FBS and Pen/Strep. The following day, 2E5 cells were used per determination point. Cells stimulated in the presence of DMSO (0.5%) or compounds (0.5% DMSO final) with anti-CD3/CD28 coated beads (Dynabeads 11452D; Invitrogen) (1:1 ratio) for 48 hours. Cytokine levels measured using Luminex platform (Millipore).

Real-Time RT-PCR

RNA was purified from cells using an RNeasy Mini Kit (Qiagen) according to the manufacturer's protocol. First-strand cDNA was synthesized using SuperScript III reverse transcriptase. Quantitative real-time PCR was performed using FastStart Universal Probe master mix (Roche) and Taqman probes for transcripts encoding the proteins IL-5, IL-13, and GAPDH, used for normalization, on the Stratagene MxPro3005p.

Intracellular Cytokine Staining

For intracellular staining, cells were restimulated with phorbol 12-myristate 13-acetate (PMA) (Sigma, 50 ng/ml) and ionomycin (Sigma, 500 ng/ml) for 5 h with the addition of Golgiplug (BD Bioscience). After restimulation the cells were washed, followed by fixation, permeabilization using Cytofix/Cytoperm Kit (554714; BD Bioscience) and stained for intracellular cytokines. to detect human IL-17A (560487; eBioscience), IFNγ (17-7319; eBioscience), IL-4 (12-7049-42; eBioscience), Foxp3 (12-4777-41; eBioscience). For Foxp3 intracellular staining, cells were fixed and permeabilized using the Foxp3/Transcription Factor Staining Kit (00-5523-00; eBioscience). Cells were acquired on the FACS Calibur (BD Bioscience) and data analyzed using FlowJo Software.

DSF Assay

BRD7/9 DSF protocol: Compounds (10 mM) or DMSO were diluted in the DSF Assay Buffer (50 mM HEPES, pH8.0, 100 mM NaCl, 0.5 mM TCEP) to generate 0.5 mM compound solution or 5% DMSO. Prepare Protein/Dye Mix (12.5× SYPRO® Orange and bromodomain protein, 6.25 mM for BRD7 and 7.5 mM for BRD9) in the DSF Assay Buffer. Transfer 3 ml of 0.5 mM compound solution or 5% DMSO to a 384-well PCR plate. Added were 12 ml of the Protein/Dye Mix to the compound plate, the plate was sealed and spun at 1000 rpm for 1 minute. The plate was run in LightCycler 480 II from 25° C. to 85° C. with a ramp rate of 0.05° C./sec. Data was analyzed with the Tm Calling module of LightCycler 480 SW 1.5.0.

### Methodology and Results

Covalent modification of histones is a fundamental mechanism of control of gene expression, and one of the major epigenetic mechanisms at play in eukaryotic cells (Kouzarides, Cell 128: 693-705 (2007)). Because distinct transcriptional states define fundamental cellular processes, such as cell type specification, lineage commitment, cell activation and cell death, their aberrant regulation is at the core of a range of diseases (Medzhitov et al., Nat. Rev. Immunol. 9: 692-703 (2009); Portela et al., Nat. Biotech. 28: 1057-1068 (2010)). A fundamental component of the epigenetic control of gene expression is the interpretation of histone modifications by proteins that harbor specialized motifs that bind to such modifications. Among them, bromodomains have evolved to bind to acetylated histones and by so doing they represent fundamental links between chromatin structure and gene transcription (Fillipakoppoulos et al., Cell 149: 214-231 (2012)). Methods of treating immune-mediated diseases by pharmacologically interfering with the bromodomain harbored in 2 proteins, BRD7 and BRD9, which may be described as BRD7/9, are described herein.

To explore if BRD7/9 bromodomains might be targets for the treatment of immune-mediated diseases, the functional impact of using potent and selective small molecule inhibitor compounds designed to bind to BRD7/9 bromodomains, thus preventing their association with acetylated histones in chromatin, was investigated. In these experiments human CD4+ T cells were used, as these cells are known to play key roles in autoimmunity and inflammation. Since small molecule inhibitors can have off-target effects, a panel of compounds from distinct chemical series with a range of biochemical potencies (Table 1, see below) was tested, to rule out such off-target effects.

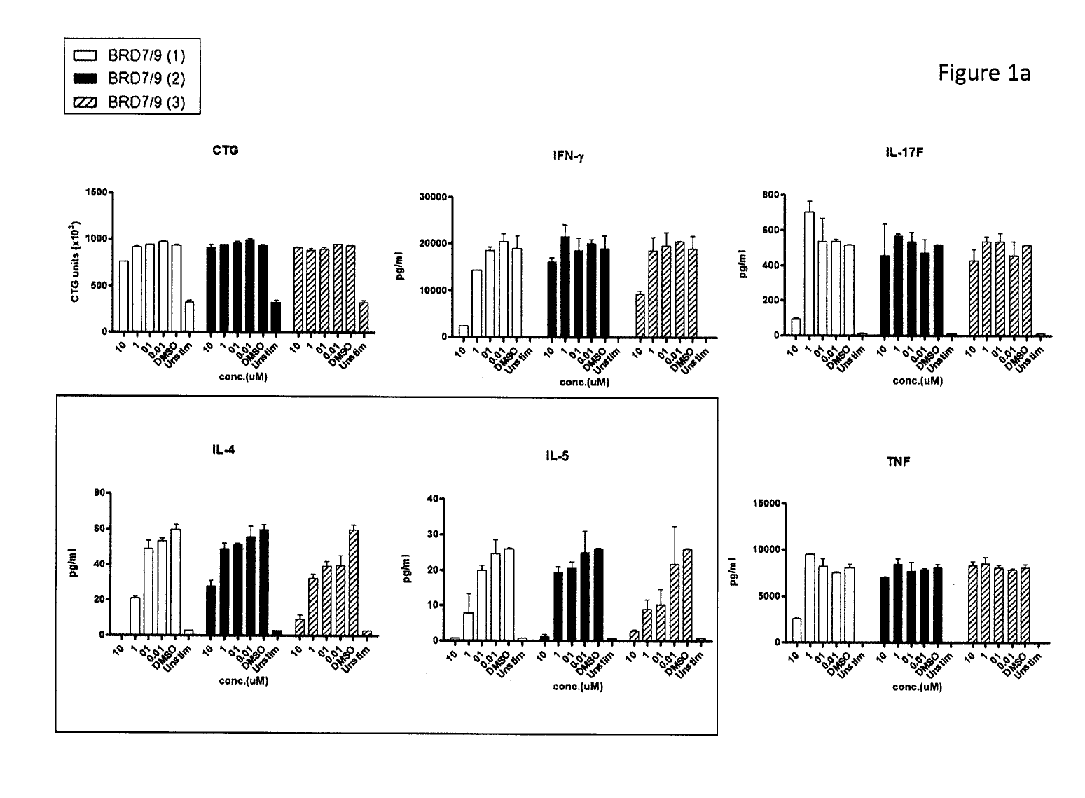

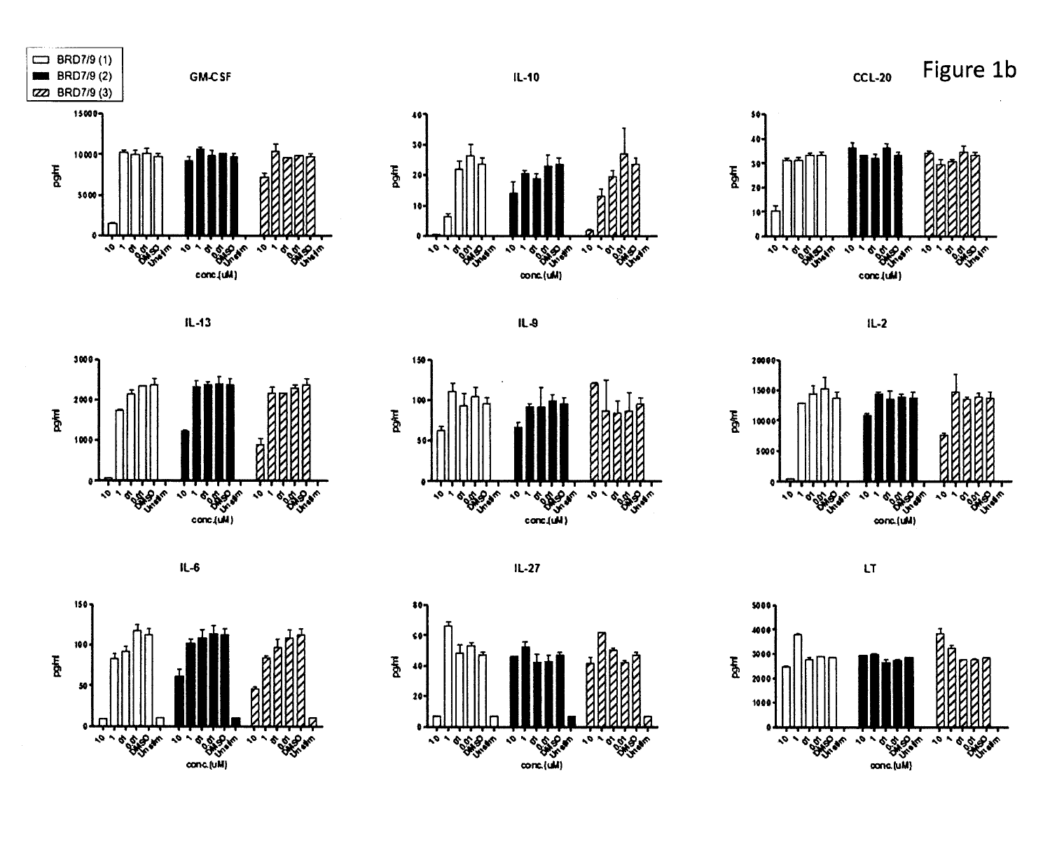

In a first set of experiments, human peripheral blood mononuclear cells (PBMC) were purified from healthy donors and cultured in the presence of PHA-p and human recombinant interleukin (IL)-2. This procedure induces activation and expansion of all CD4+T cells present in the PBMC preparation, rendering a highly enriched mixture of pre-activated T cells representative of all subsets present in the original PBMC preparation (T blasts). Activation of such cells through the T cell receptor (TCR) using a combination of anti-CD3 and anti-CD28 antibodies results in the expression and secretion of cytokines that can be readily measured in the culture medium. As shown in FIG. 1a, the BRD7/9 inhibitors BRD7/9(1), BRD7/9(2) and BRD7/9(3) were shown to reduce, in a dose-dependent manner, the production of IL-4 and IL-5, as measured using the Luminex platform, but not cytokines representative of other subsets, such as interferon-gamma (IFN-gamma) or IL-17F. Tumor necrosis factor (TNF)-alpha, a generic pro-inflammatory cytokine was not inhibited. Importantly, cell viability (measured as ATP production) was not affected by any of the compounds (FIG. 1a). Further, the impact of these inhibitors on a wide panel of 9 additional cytokines was investigated. No significant and consistent effect on any of those cytokines was found across compounds (FIG. 1b).

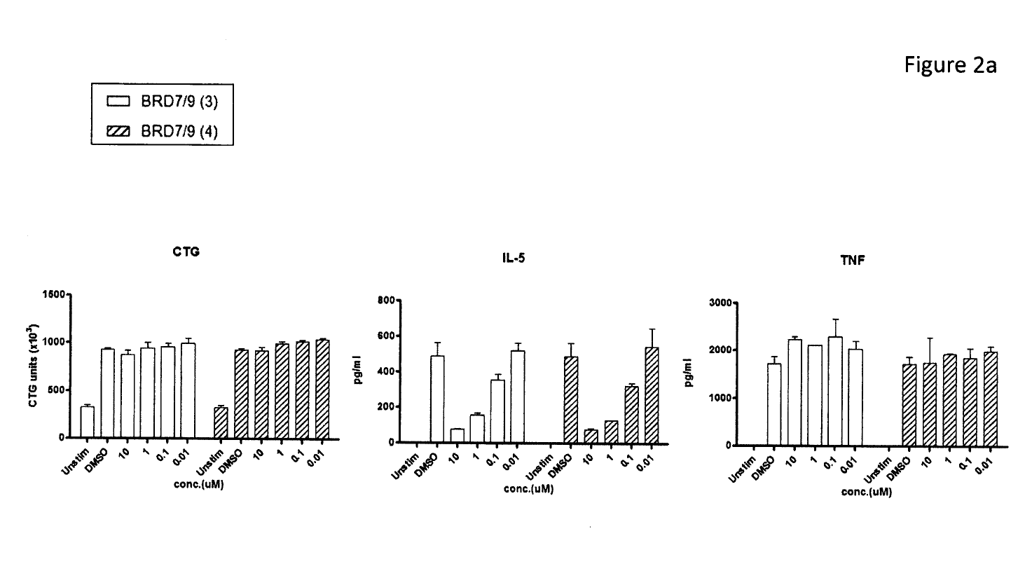

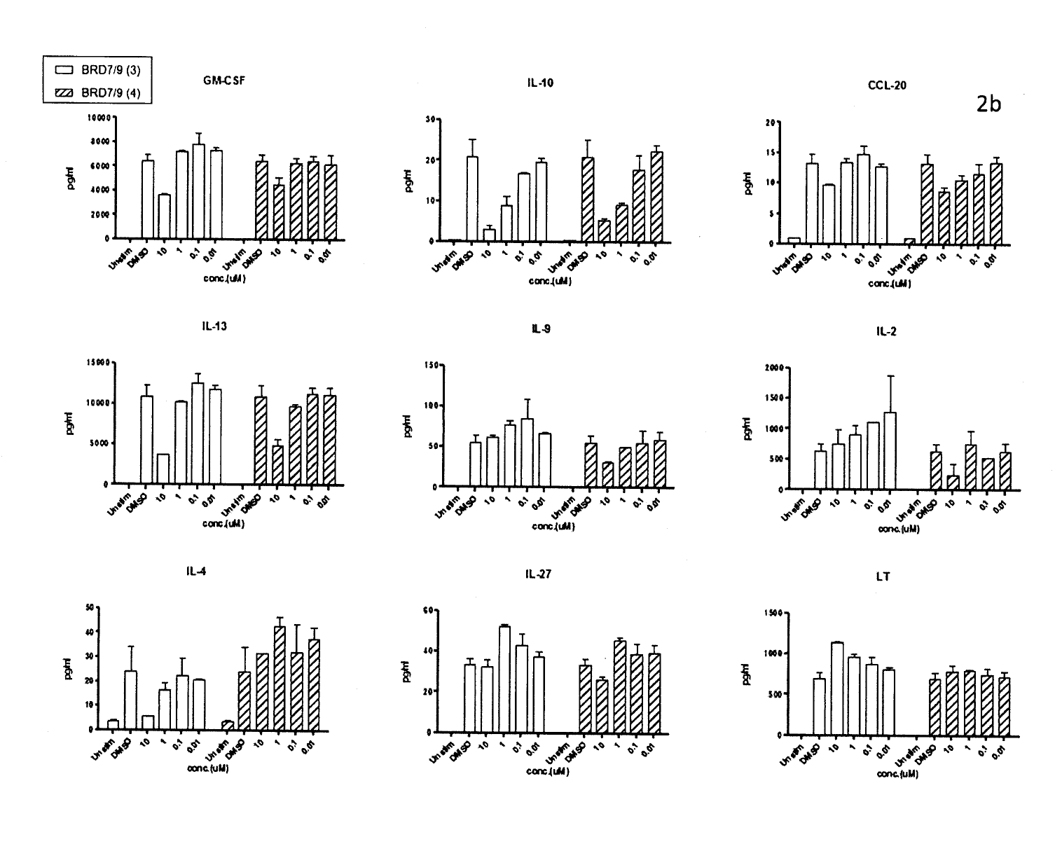

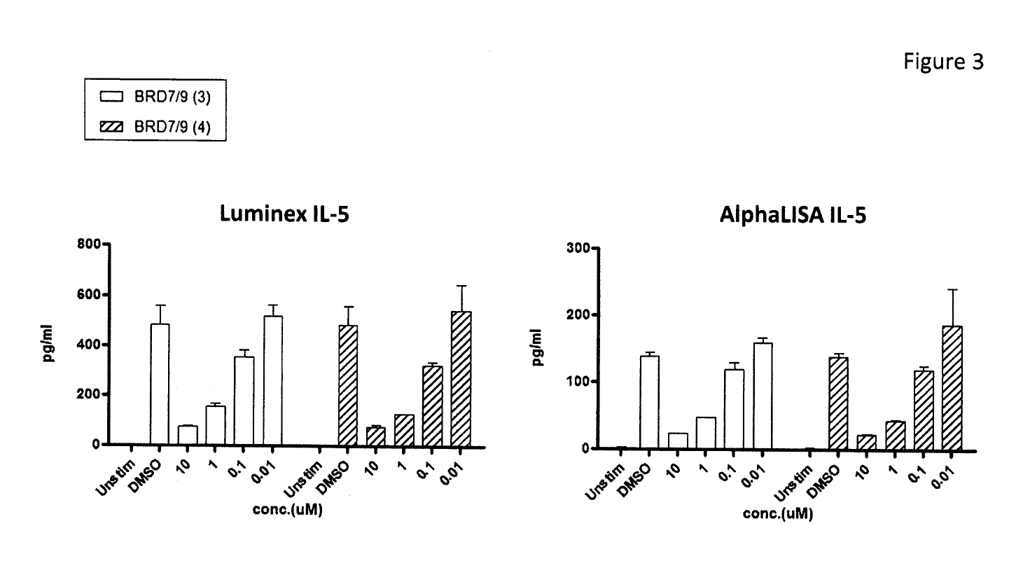

IL-4 and IL-5 (together with IL-13) are cytokines selectively produced by the T helper (Th) type 2 subset of CD4+ T cells and are known to mediate allergic responses such as asthma and allergic rhinitis (Fanta, Asthma. New Eng. J. Med. 360: 1002-1014 (2009)). Because the BRD7/9 inhibitors consistently and selectively inhibited these Th2 cytokines, it was proposed that BRD7/9 inhibition could be an efficient way to suppress cytokine production from Th2 cells. To test this hypothesis, Th2 cells were prepared from purified naïve human CD4+ T cells. These naïve T cells can be identified by their surface expression of the marker CD45RA, and then differentiated in vitro with a standard and well established mix of cytokines, as described in the Methods section. As shown in FIG. 2a, the BRD7/9 inhibitors BRD7/9(3) and BRD7/9(4) were shown to reduce, in a dose-dependent manner, the production of IL-5. Consistent with the data presented in FIG. 1, TNF-alpha or cell viability were not affected by any of the compounds. On a wide panel of 9 additional cytokines, and consistent with the data described in FIG. 1b, no impact of any of the compounds tested was found, with the exception of IL-10, another Th2-enriched cytokine (FIG. 2b). As shown in FIG. 3, the BRD7/9 inhibitors BRD7/9(3) and BRD7/9(4) were also shown to reduce, in a dose-dependent manner, the production of IL-5 as measured using the Luminex and the AlphaLISA platforms.

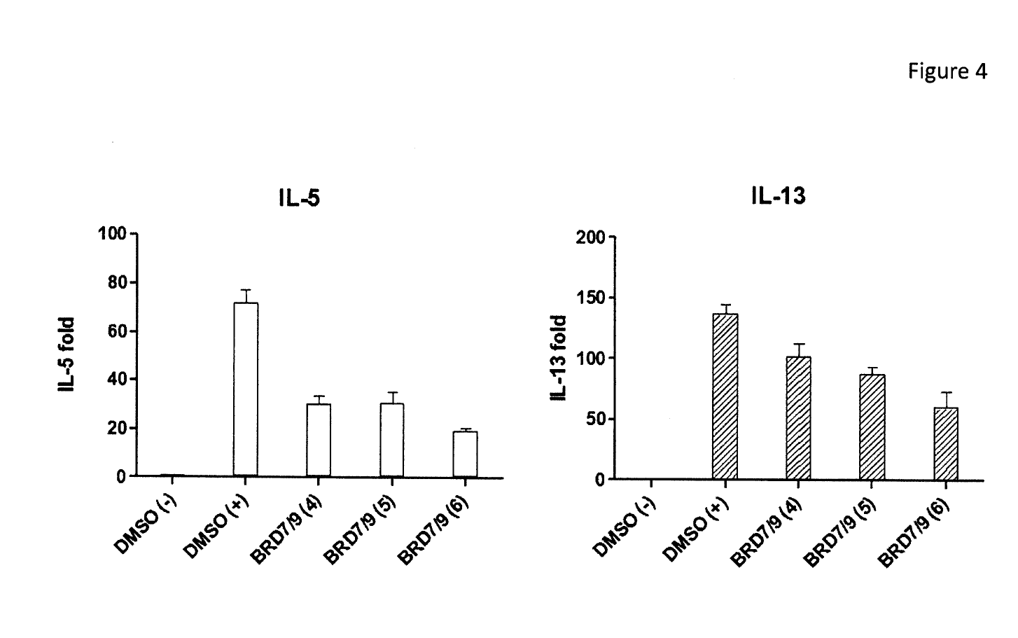

Because the data presented herein demonstrates an important role for BRD7/9 bromodomains in Th2 cytokine production, and because bromodomains are protein motifs that mediate binding to chromatin, it was hypothesized that the BRD7/9 bromodomain inhibition could impact transcription of genes encoding Th2 cytokines. As shown in FIG. 4, the BRD7/9 inhibitors BRD7/9(4), BRD7/9(5) and BRD7/9(6) were shown to reduce gene transcript accumulation of IL-5 and IL-13 (canonical Th2 cytokines).

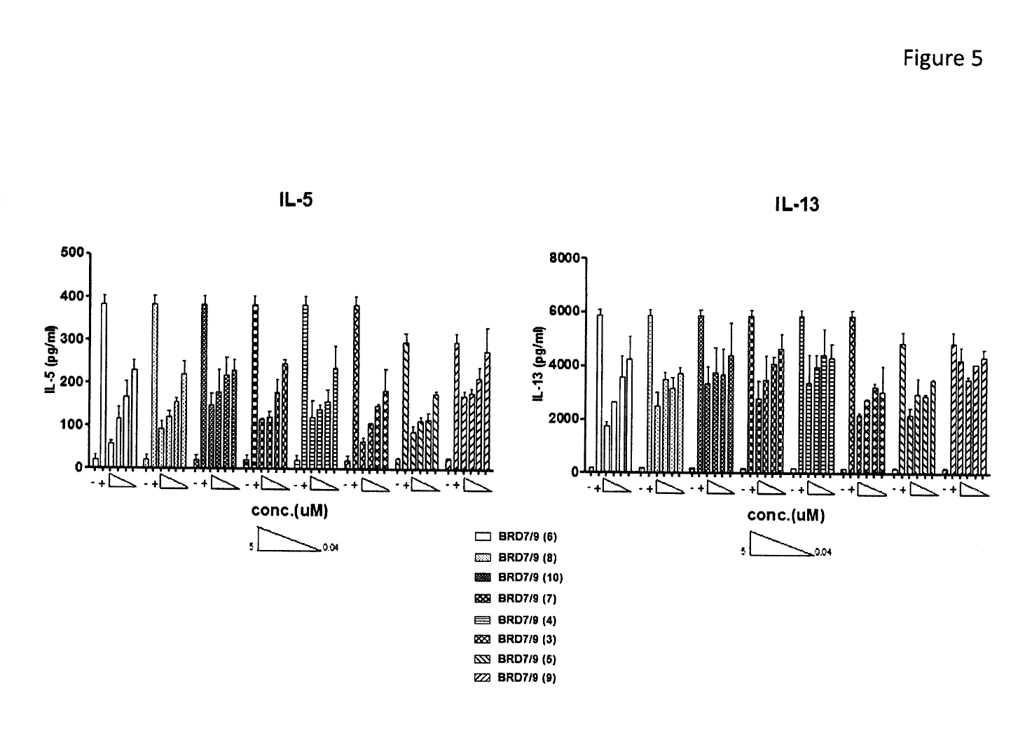

To further demonstrate that the impact on Th2 cytokine production was mediated by BRD7/9 bromodomain inhibition, an additional set of experiments was conducted in which the effect of a large panel of inhibitors with different biochemical potencies was investigated. As shown in FIG. 5, all compounds had a selective impact on IL-5 and IL-13 production. This effect was dose-dependent, and had a magnitude that was proportional to their biochemical potencies.

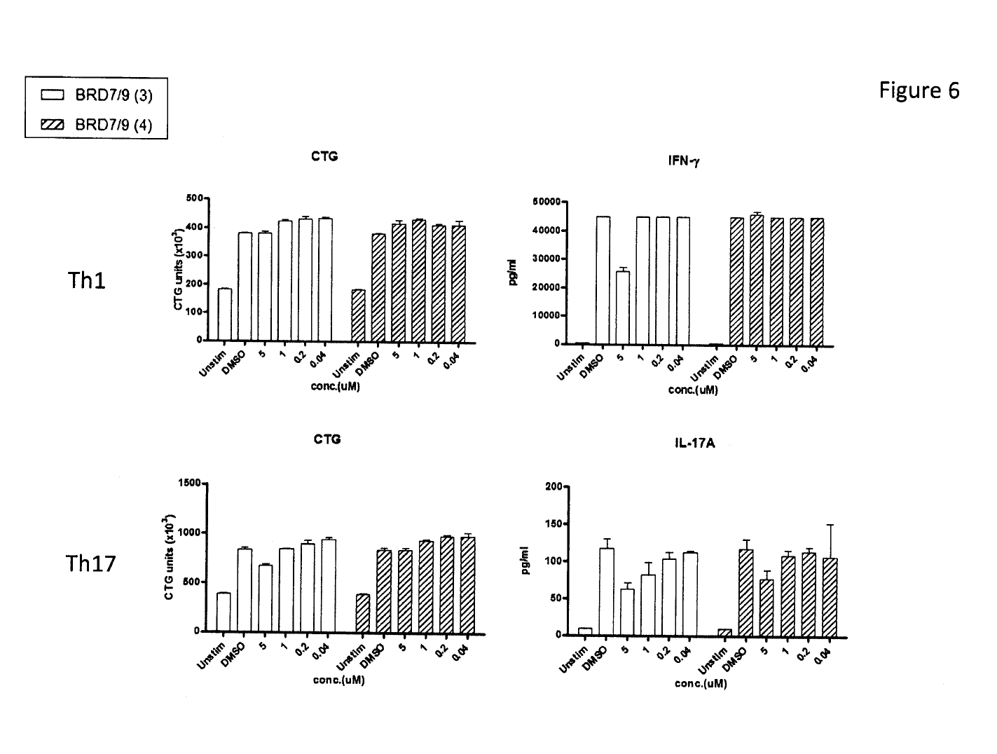

Whether the observed effects of BRD7/9 bromodomain inhibition in Th2 cells were selective for this lineage was investigated. Naïve CD4+T cells were differentiated into Th1 and Th17 cells, and the effects of the inhibitors on their cytokine profile were investigated, in particular on their profile of canonical cytokines: interferon-gamma (IFN-γ) in Th1 cells, and IL17A in Th17 cells. As shown in FIG. 6, BRD7/9(3) and BRD7/9(4) inhibitors had no impact on IFN-γ in Th1 cells. Similarly, no effect of the BRD7/9(4) inhibitor on IL-17A was found in Th17 cells, and only a very modest effect of BRD7/9(3) on that cytokine.

Taken together, the data described thus far demonstrate that inhibition of BRD7/9 bromodomains result in the selective and dose-dependent suppression of the Th2-specific cytokines IL-4, IL-5 and IL-13.

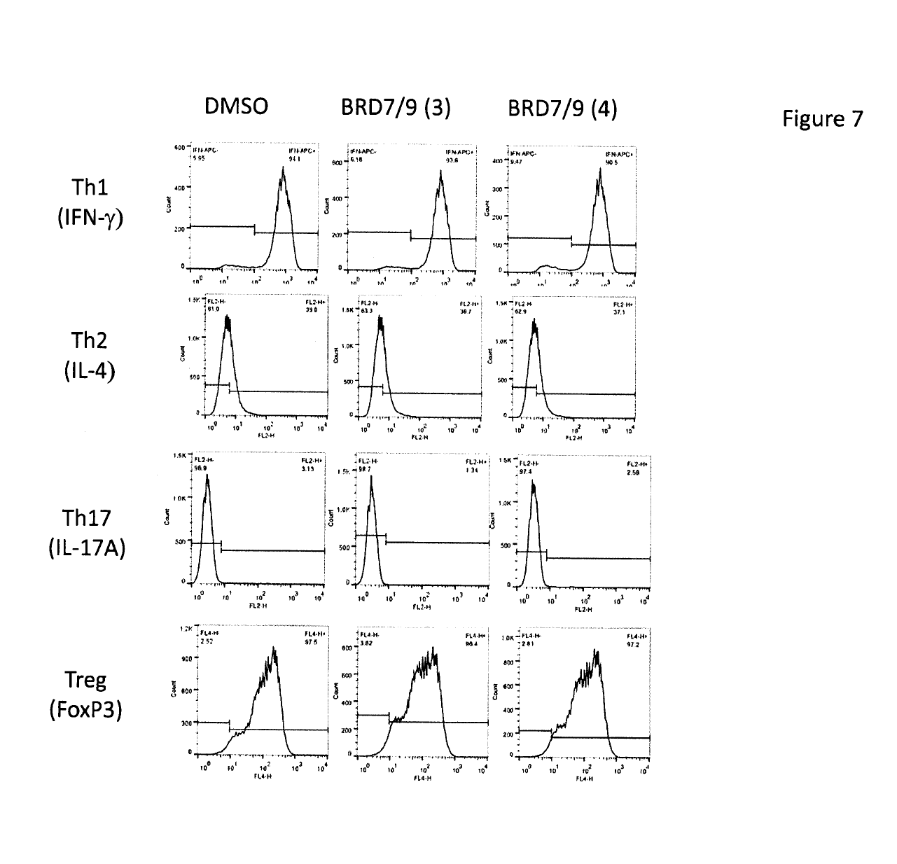

Whether BRD7/9 bromodomain inhibition had any effect in the differentiation of any of the major CD4+T cell subsets, Th1, Th2, Th17 and T regulatory (Treg) cells, was investigated. With that purpose, human naïve CD4+ T cells were differentiated with the appropriate standard and well-established cocktails of cytokines described in the Methods section, and the effect of the inhibitors BRD7/9(3) and BRD7/9(4) was explored. Fluorescence-activated cell sorting (FACS) to enumerate IFN-γ-expressing cells (Th1), IL-4-expressing cells (Th2), IL-17A-expressing cells (Th17) and FoxP3-expressing cells (Tregs) was used to assess differentiation. As shown in FIG. 7, BRD7/9 bromodomain inhibition during differentiation of naïve T cells into Th1, Th2, Th17 or Tregs had no functional impact. Specifically, no significant effect on the number of IFN-γ-expressing cells in the Th1 cultures, or IL-4-expressing cells in the Th2 cultures, or IL-17A-expressing cells in the Th17 cultures, or FoxP3-expressing cells in the Treg cultures, was detected.

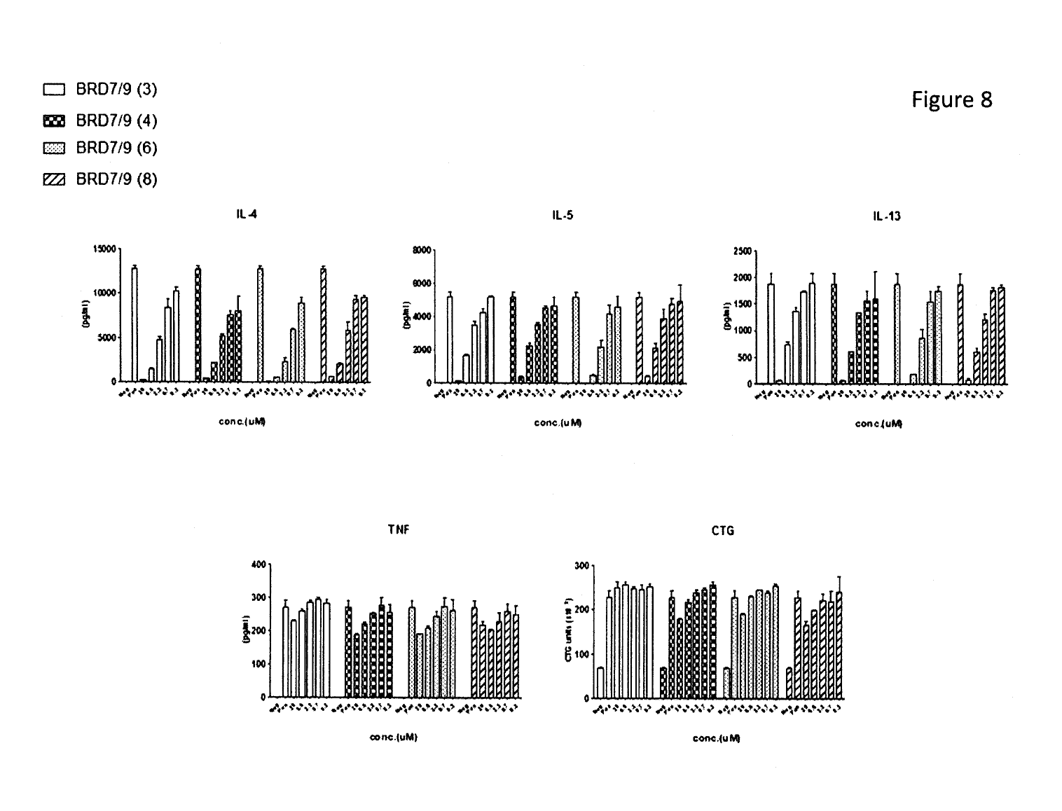

Finally, whether the critical role of BRD7/9 bromodomains uncovered in the studies reported herein was conserved in other species, such as mouse, was investigated. Mouse naïve T cells were differentiated into Th2 cells for 4 days, and then re-stimulated with anti-CD3 and anti-CD28 for 40 hours. As shown in FIG. 8, BRD7/9 bromodomain inhibition resulted in a significant and dose-dependent inhibition of the canonical Th2 cytokines IL-4, IL-5 and IL-13, while the generic pro-inflammatory cytokine TNF-alpha was only modestly affected. None of the compounds exhibited any significant effect on cell viability (FIG. 8).

In summary, as described herein, it has been demonstrated that BRD7/9 bromodomains play an unexpected but critical role in the expression of human and mouse Th2 cytokines, in particular IL-4, IL-5 and IL-13, but they are dispensable for the expression of other cytokines. Moreover, BRD7/9 bromodomain inhibition has no effect on the differentiation of any T cell subset studied (Th1, Th2, Th17 and Treg). Because Th2 cytokines mediate allergic diseases, an effective way to treat such diseases, that include, but are not limited to, asthma, eosinophilic severe asthma, eosinophilic syndromes, allergic rhinitis and allergic dermatitis, among others, has been discovered.

Table 1, below, provides the biochemical data for the BRD7/9 compounds. The IC50 values of Table 1 were generated using the AlphaLISA assay described below.

AlphaLISA assay for measuring IC50 values:

| | |
|---|---|
| Plate type | ProxiPlate 384 Plus |
| Assay Rx volume (uL) | 19 |
| Top concentration (uM) | 80 |
| DMSO backfill (nL) | 220 |
| IC50 control compound | BRD7/9 compound 1-10 |
| IC50 cont top conc. (uM) | 80 |
| MIN control | DMSO/no peptide |
| MAX control | DMSO |

The following reaction buffers were prepared:

### 1× Reaction Buffer

| Final | | Working | | Stock | | Reagent |
|---|---|---|---|---|---|---|
| 50 | mM | 50 | mM | 1000 | mM | HEPES |
| | | | | | | pH 7.5 |
| 1 | mM | 1 | mM | 500 | mM | TCEP |
| 0.069 | mM | 0.069 | mM | 83.47 | mM | Brij-35 |
| 150 | mM | 30 | mM | 5000 | mM | NaCl |
| 0.1 | mg/mL | 0.1 | mg/mL | 10 | mg/mL | BSA |

MilliQ Water

### 3× Reaction Buffer Solution A-6.3 uL

| Final | | Working | | Stock | | Reagent |
|---|---|---|---|---|---|---|
| 0.050 | uM | 0.09 | uM | 185.68 | uM | BRD9 |

1X Reaction Buffer

### 3× Binder Solution B-6.3 uL

| Final | | Working | | Stock | | Reagent |
|---|---|---|---|---|---|---|
| 0.003 | uM | 0.0048 | uM | 100 | uM | 100 uM stock H4-tetraacetyl |
| | | | | | | peptide (New England |
| | | | | | | Peptide (NEP2069-11/13)* |

1X Reaction Buffer
LCBiot-AASGRG(Kac)GG(Kac)GLG(Kac)GGA(Kac)RHRK-amide

### 3× Beads C-6.3 uL

| Final | | Working | | Stock | | Reagent |
|---|---|---|---|---|---|---|
| 15 | ug/mL | 45 | ug/mL | 5000 | ug/mL | Strep Acceptor Bead |
| | | | | | | (Perkin-AL125C) |
| 15 | ug/mL | 45 | ug/mL | 5000 | ug/mL | Nickel Donor Bead |
| | | | | | | (Perkin AS 10 ID) |

1X Reaction Buffer

### 1× Reaction Buffer

Solution A (6.3 uL per well) and Solution B (6.3 uL per well) were combined and incubated for 20 minutes at room temperature. In dim light, 6.3 uL of the Beads C solution were added. The resulting solutions were covered with a microplate TopSeal and incubated for 90 minutes in the dark at room temperature. The plates were read with Envision (Using protocol: AlphaLisa_ProxiPlate Flatfield corrected). Data was analyzed manually or using Activity base (Abase) or manually. For manually processed data, IC50's were generally derived using GraphPad Prism 5 and a 4 parameter dose-response fit. Results are provided in Table 1.

TABLE 1
List of BRD7/9 compounds used in this study including
their biochemical in vitro potencies.

| | BRD9 | | |
|---|---|---|---|
| | Biochemical | | |
| | assay | BRD9 DSF | BRD7 DSF |
| | (alphascreen) | deltaTm | delta Tm |
| Comp ID | (uM) | (degrees C.) | (degrees C.) |
| BRD7/9 (1) | 0.0434 | | |
| BRD7/9 (2) | 0.0529 | | |
| BRB7/9 (3) | 0.0352 | 10.9 | 10.5 |
| BRD7/9 (4) | 0.0251 | 7.1 | |
| BRD7/9 (5) | 0.0307 | 10.4 | 10.1 |
| BRD7/3 (6) | 0.0456 | 7.2 | 6.5 |
| BRD7/9 (7) | 0.0377 | 10.7 | 10.8 |
| BRD7/9 (8) | 0.0399 | 10.1 | 8.2 |
| BRD7/9 (9) | 0.0644 | 7 | 5.4 |
| BRB7/9 (10) | 0.0359 | 3.2 | 5.4 |

### Example 2

### Assay for Visualizing BRD9 Localization Upon Inhibitor Treatment

A stable cell line carrying an inducible BRD9 fluorescent fusion protein was seeded at 20,000 cells per well. Expression of the fusion was induced by addition of 2 μg/mL doxycyclin for 16 h at 37° C. Test inhibitors were then added in medium lacking doxycyclin for 60 min at room temperature. Cells were fixed with 4% PFA and then Hoechst stained for 30 min Images were acquired in both green and blue channels. Green BRD9 puncta of a minimum chosen size were identified as “pits” and response to inhibitors was quantified as “pits per cell”.

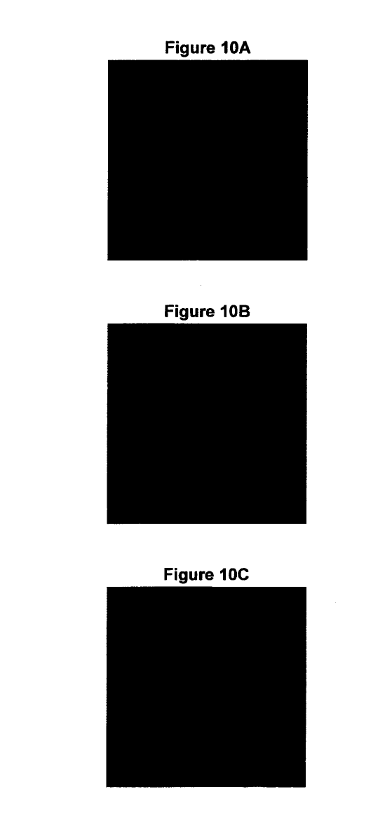

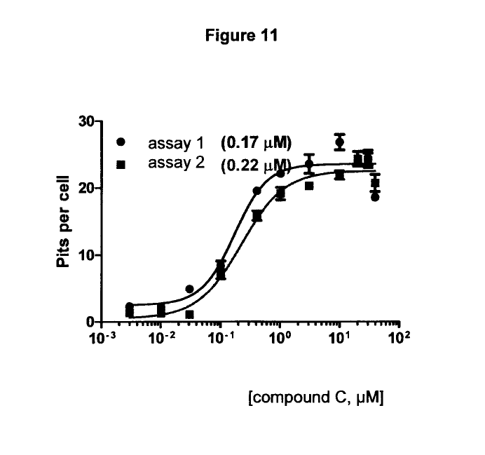

FIGS. 10A-10C demonstrate that BRD9 fusion protein is localized predominantly to chromatin in the absence of inhibitor and is found in large puncta upon compound addition. FIG. 11 depicts dose-response curves generated using compound BRD7/9 (8) in the presence of BRD9.

### Example 3

### Determination of Selectivity for BRD9 Over Other Bromodomains

Compound BRD7/9 (8) was tested for binding to various bromodomains by AlphaLisa using a protocol similar to that described above for BRD9 (Example 1). The selectivity ratio in Table 2 is the IC50 for the indicated bromodomain divided by the IC50 for BRD9.

TABLE 2
Binding Selectivity

| Bromodomain | Selectivity Ratio |
|---|---|
| BRD4_1 | 240 |
| BRD8 | >875 |
| BRPF1 | >985 |
| CBP | 480 |
| CECR2 | 982 |
| PCAF | >985 |
| TAF1_2 | 480 |
| TRIM24 | 1000 |

Although the invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. A method for treating a TH2 disease in a mammal comprising administering a therapeutically effective amount of an inhibitor of BRD7 or BRD9 to the mammal.

2. The method of claim 1 wherein the inhibitor inhibits BRD7.

3. The method of claim 1 wherein the inhibitor inhibits BRD9.

4. The method of claim 1 wherein the inhibitor inhibits BRD7 and BRD9.

5. The method of any one of claims 1-4 wherein the agent binds to a bromodomain.

6. The method of any one of claims 1-4 wherein the agent binds to isoform 1 of BRD7 or BRD9.

7. The method of any one of claims 1-4 wherein the agent binds to isoform 1 of BRD7 and BRD9.

8. The method of any one of claims 1-7 wherein the inhibitor is an siRNA, shRNA, small molecule, or a macrocycle.

9. The method of any one of claims 1-8 wherein the TH2 disease is an immune-related disease or disorder associated with excess TH2 cytokine.

10. The method of any one of claims 1-8 wherein the TH2 disease is selected from atopic dermatitis, allergies, allergic rhinitis, asthma, chronic obstructive pulmonary disease, hypereosinophilic syndrome, eosinophilic esophagitis, Churg-Strauss syndrome, and nasal polyposis.

11. The method of any one of claims 1-8 wherein the TH2 disease is a respiratory disorder.

12. The method of any one of claims 1-8 wherein the TH2 disease is selected from asthma; bronchitis; chronic obstructive pulmonary disease; and conditions involving airway inflammation.

13. The method of any one of claims 1-8 wherein the TH2 disease is an eosinophilic disorder.

14. The method of claim 13 wherein the eosinophilic disorder is selected from asthma, atopic asthma, atopic dermatitis, allergic rhinitis, non-allergic rhinitis, chronic eosinophilic pneumonia, allergic bronchopulmonary aspergillosis, coeliac disease, Churg-Strauss syndrome, eosinophilic myalgia syndrome, hypereosinophilic syndrome, oedematous reactions, helminth infections, onchocercal dermatitis and Eosinophil-Associated Gastrointestinal Disorders.

15. The method of claim 13 wherein the eosinophilic disorder is selected from eosinophilic esophagitis, eosinophilic gastritis, eosinophilic gastroenteritis, eosinophilic enteritis and eosinophilic colitis, nasal micropolyposis and polyposis, aspirin intolerance, asthma and obstructive sleep apnoea.

16. The method of claim 1 wherein the inhibitor is at least 5 fold selective for bromodomain-containing protein BRD7 over other bromodomain-containing proteins.

17. The method of claim 1 wherein the inhibitor is at least 5 fold selective for bromodomain-containing protein BRD9 over other bromodomain-containing proteins.

18. The method of claim 1 wherein the inhibitor is at least 5 fold selective for bromodomain-containing proteins BRD7 and BRD9 over other bromodomain-containing proteins.

19. The method of claim 1 wherein the inhibitor binds is least 5 fold selective for BRD7 over other bromodomains.

20. The method of claim 1 wherein the inhibitor binds is least 5 fold selective for BRD9 over other bromodomains.

21. The method of claim 1 wherein the inhibitor binds is least 5 fold selective for BRD7 and BRD9 over other bromodomains.

22. The method of any one of claims 1-21, wherein the inhibitor inhibits the production of IL-4, IL-5, or IL-13.

23. An inhibitor of BRD7 or BRD9 for the prophylactic or therapeutic treatment of a TH2 disease.

24. The inhibitor of claim 23 wherein the inhibitor inhibits BRD7.

25. The inhibitor of claim 23 wherein the inhibitor inhibits BRD9.

26. The inhibitor of claim 23 wherein the inhibitor inhibits BRD7 and BRD9.

27. The inhibitor of any one of claims 23-26 wherein the agent binds to a bromodomain.

28. The inhibitor of any one of claims 23-26 wherein the agent binds to isoform 1 of BRD7 or BRD9.

29. The inhibitor of any one of claims 23-26 wherein the agent binds to isoform 1 of BRD7 and BRD9.

30. The inhibitor of any one of claims 23-29 wherein the inhibitor is an siRNA, shRNA, small molecule, or a macrocycle.

31. The inhibitor of any one of claims 23-30 wherein the TH2 disease is an immune-related disease or disorder associated with excess TH2 cytokine.

32. The inhibitor of any one of claims 23-30 wherein the TH2 disease is selected from atopic dermatitis, allergies, allergic rhinitis, asthma, chronic obstructive pulmonary disease, hypereosinophilic syndrome, eosinophilic esophagitis, Churg-Strauss syndrome, and nasal polyposis.

33. The inhibitor of any one of claims 23-30 wherein the TH2 disease is a respiratory disorder.

34. The inhibitor of any one of claims 23-30 wherein the TH2 disease is selected from asthma; bronchitis; chronic obstructive pulmonary disease; and conditions involving airway inflammation.

35. The use of an inhibitor of BRD7 or BRD9 to prepare a medicament for the treatment of a TH2 disease.

36. The use of claim 35 wherein the inhibitor inhibits BRD7.

37. The use of claim 35 wherein the inhibitor inhibits BRD9.

38. The use of claim 35 wherein the inhibitor inhibits BRD7 and BRD9.

39. The use of any one of claims 35-38 wherein the agent binds to a bromodomain.

40. The use of any one of claims 35-38 wherein the agent binds to isoform 1 of BRD7 or BRD9.

41. The use of any one of claims 35-38 wherein the agent binds to isoform 1 of BRD7 and BRD9.

42. The use of any one of claims 35-41 wherein the inhibitor is an siRNA, shRNA, small molecule, or a macrocycle.

43. The use of any one of claims 35-42 wherein the TH2 disease is an immune-related disease or disorder associated with excess TH2 cytokine.

44. The use of any one of claims 35-42 wherein the TH2 disease is selected from atopic dermatitis, allergies, allergic rhinitis, asthma, chronic obstructive pulmonary disease, hypereosinophilic syndrome, eosinophilic esophagitis, Churg-Strauss syndrome, and nasal polyposis.

45. The use of any one of claims 35-42 wherein the TH2 disease is a respiratory disorder.

46. The use of any one of claims 35-42 wherein the TH2 disease is selected from asthma; bronchitis; chronic obstructive pulmonary disease; and conditions involving airway inflammation.

47. A pharmaceutical composition for use in the treatment of a TH2 disease, comprising an inhibitor of BRD7 or BRD9 and a pharmaceutically acceptable carrier.

48. The pharmaceutical composition of claim 47 wherein the inhibitor inhibits BRD7.

49. The pharmaceutical composition of claim 47 wherein the inhibitor inhibits BRD9.

50. The pharmaceutical composition of claim 47 wherein the inhibitor inhibits BRD7 and BRD9.

51. The pharmaceutical composition of any one of claims 47-50 wherein the agent binds to a bromodomain.

52. The pharmaceutical composition of any one of claims 47-50 wherein the agent binds to isoform 1 of BRD7 or BRD9.

53. The pharmaceutical composition of any one of claims 47-50 wherein the agent binds to isoform 1 of BRD7 and BRD9.

54. The pharmaceutical composition of any one of claims 47-53 wherein the inhibitor is an siRNA, shRNA, small molecule, or a macrocycle.

55. A method of identifying a compound useful for treating a TH2 disease comprising determining whether the compound inhibits BRD7 or BRD9.

56. The method of claim 55 comprising determining whether the compound inhibits BRD7.

57. The method of claim 55 comprising determining whether the compound inhibits BRD9.

58. The method of any one of claims 55-57 wherein the determining comprises contacting the compound with BRD7 or BRD9 and measuring whether the activity of the BRD7 or BRD9 decreases.

59. The method of claim 58 wherein the compound is contacted with BRD7.

60. The method of claim 58 wherein the compound is contacted with BRD9.

61. The method of claim 58 wherein the measuring is carried out as described in Example 1.

62. A method for inhibiting the production of IL-4, IL-5, or IL-13 in a mammal comprising administering an inhibitor of BRD7 or BRD9 to the mammal.

63. The method of claim 62 wherein the inhibitor inhibits BRD7.

64. The method of claim 62 wherein the inhibitor inhibits BRD9.

65. The method of claim 62 wherein the inhibitor inhibits BRD7 and BRD9.

66. The method of any one of claims 62-65 wherein the inhibitor binds to a bromodomain.

67. The method of any one of claims 62-66 wherein the inhibitor binds to at least isoform 1 of BRD7 or BRD9.

68. The method of any one of claims 62-66 wherein the inhibitor binds to at least isoform 1 of BRD7 and BRD9.

69. The method of any one of claims 62-68 wherein the inhibitor is an siRNA, shRNA, small molecule, or a macrocycle.

70. The method of any one of claim 1-22 or 62-69 wherein the mammal is a mammal in need of such treatment.

71. The method, inhibitor, use or composition of any one of claims 1-70, wherein the inhibitor has an IC₅₀ against BRD7 and/or BRD9 of less than 10 μM, e.g., less than 1 μM, e.g., less than 100 nM, e.g., less than 10 nM, e.g., less than 1 nM.

72. The method, inhibitor, use or composition of any one of claims 1-71, wherein the inhibitor has a binding affinity against BRD7 and/or BRD9 with a K_{d} of less than 1,000 nm, e.g., less than 500 nM, e.g., less than 100 nM, e.g., less than 50 nM.

73. The method, inhibitor, use or composition of any one of claims 1-71, wherein the inhibitor has a binding affinity against BRD7 and/or BRD9 of between 500 nM to 1 pM.
